(19) United States
(12) Patent Application Publication
(10) Pub. No.: US 2012/0157422 A1
(43) Pub. Date: Jun. 21, 2012

(54) MARKER

(75) Inventors: Lucilla Poston, London (GB); Paul Seed, London (GB); Gillian Lachelin, Ockham, Surrey (GB)

(73) Assignees: UCL BUSINESS PLC, London (GB); KING'S COLLEGE LONDON, London (GB)

(21) Appl. No.: 13/263,159

(22) PCT Filed: Apr. 6, 2010
(86) PCT No.: PCT/GB2010/000690

§ 371 Date: Feb. 28, 2012

(30) Foreign Application Priority Data
Apr. 6, 2009 (GB) 0905964.3

(51) Int. Cl. A61K 31/57; G01N 33/74

(52) U.S. Cl. 514/177; 436/501

(57) ABSTRACT

The invention provides a method for identifying whether a pregnant subject has an increased risk of preterm labour comprising measuring the progesterone concentration in a sample obtained from the subject, wherein a reduced progesterone concentration is indicative of an increased risk of preterm labour and delivery.

### FIELD OF THE INVENTION

The invention relates to a method of identifying pregnant women at risk of preterm labour. In particular, the invention relates to a diagnostic marker found in saliva samples which can be used to identify women likely to deliver preterm.

### BACKGROUND TO THE INVENTION

The incidence of preterm labour in developed countries has remained unchanged over several decades, complicating approximately 7% of deliveries in developed countries. Whilst survival rates have improved dramatically, morbidity remains high. Identification of those women who are at greatest risk of preterm labour and delivery is inaccurate and still largely based on clinical history.

Labour in sheep is preceded by a decrease in progesterone and an increase in oestradiol (E2) plasma concentrations which result from conversion of progesterone to E2, mediated by placental 17α hydroxylase. 17α hydroxylase is present in very low levels, if at all, in the human placenta and there is no evidence for a decrease in progesterone or an increase in E2 before the onset of normal labour in women, although the concept of ‘functional progesterone withdrawal’ has been supported by investigations of the progesterone receptor and its isoforms.

Concentrations of steroids in saliva reflect the unbound, unconjugated and therefore the biologically active fraction of the plasma hormone profile. Since saliva specimens are easy to collect and store, measurement of saliva hormones can be readily introduced into clinical practice when found to be of value.

A number of previous studies have suggested that an increase in the saliva oestriol (E3) and the E3 to progesterone ratio may be an indicator of impending delivery. Nevertheless, it would be useful to identify further indicators of the likelihood of preterm labour to assist in accurately identifying those women who might be at risk and those who might benefit from or respond to progesterone treatment.

### SUMMARY OF THE INVENTION

According to the invention, there is provided a method for identifying whether a pregnant animal has an increased risk of preterm labour and subsequent delivery comprising measuring the progesterone in a sample obtained from the subject, wherein a reduced progesterone concentration is indicative of an increased risk of preterm labour.

The animal is preferable a mammal, especially a primate. In one embodiment, the animal is a human.

When the animal is a human, the term “preterm labour” preferably means spontaneous labour at less than 37 weeks' completed gestation. Preferably, it means at between 26 and 37 weeks' completed gestation, especially between 30 and 37 weeks' completed gestation. Gestational age may be assessed using the last menstruation date and ultrasound examination.

The sample may be any sample obtainable from the subject such as plasma, but is preferably a secreted sample, especially saliva or vaginal secretions. It is particularly useful to use saliva samples as these can be conveniently self collected.

The concentration of progesterone in the sample may be measured by any known means. Preferably the concentration is measured by radioimmunoassay.

The term “reduced progesterone concentration” preferably means that the concentration of progesterone in the sample is lower than the expected progesterone level for that stage of gestation.

The expected level of progesterone may be identified in a number of ways. For example, the sample may be compared with a comparable sample from another animal. If the progesterone level is lower than that of the comparable sample, the level may be considered to be reduced. The sample may be compared with a standard level or range of levels appropriate for the sample type and the gestation at which the sample is collected. Reference ranges may be established by testing a large number of comparable samples from a number of animals over a range of gestational ages. Such reference ranges may allow the determination of the distribution of progesterone values at a given gestation, and in particular of the centiles of the distribution. There are a number of ways of determining such centiles, well known to those familiar with the art. Comparable samples are the same type of sample as each other. For example, comparable samples are taken from animals of the same species. Equally, saliva samples should be compared with other saliva samples, urine samples with other urine samples etc.

When compared with a sample from another animal or with a standard level, the reduction in progesterone concentration is preferably a reduction of at least 10%, preferably at least 25%, more preferably at least 50%. Alternatively, where centiles have been determined, values below the 5^{th} centile, preferably the 1^{st} centile more preferably the 0.1^{th} centile, may define a reduction in progesterone concentration.

Alternatively, the sample may be compared with a projected progesterone level, established by testing the same subject at an earlier date and predicting the progesterone level that should be reached by the relevant stage of gestation.

The inventors have noted that progesterone levels rise progressively during normal pregnancy with labour onset at or near term, but that the rate of rise in the level of progesterone is reduced before preterm labour. Accordingly, if the level of progesterone has not increased, or increased by less than the expected amount, between two preterm tests taken a number of days apart, the woman can be identified as being at risk of preterm labour. When testing a number of samples from the same woman, the woman can preferably be identified as being at risk of preterm labour if the increase in progesterone levels is less than 5%, more preferably less than 3% per week, or where there is evidence of a fall in progesterone levels.

When testing samples from the same woman, the samples may be obtained daily, every two or three days or weekly, for example.

The method of identifying subjects at risk may be used in combination with other methods that identify subjects at risk. Such methods may include comparing the E3 to progesterone ratios in samples with that of other comparable samples, either from the same subject, different subjects or with an established standard ratio, a rise in that ratio being indicative of an increased risk

The method of the invention may also be used to identify subjects that may benefit from treatment with progesterone. It may be advantageous to administer progesterone to an individual at risk of preterm labour.

In order to identify subjects at risk and to be able to take action to delay or avoid preterm labour, it is advantageous to test samples taken early on during pregnancy. In human women, it is preferable to test samples taken at between 15 to 16 weeks and 30 to 34 weeks of gestation, more preferably between 16 and 28 weeks, even more preferably between 16 and 24 weeks.

The invention will now be described in detail, by way of example only, with reference to the figures in which

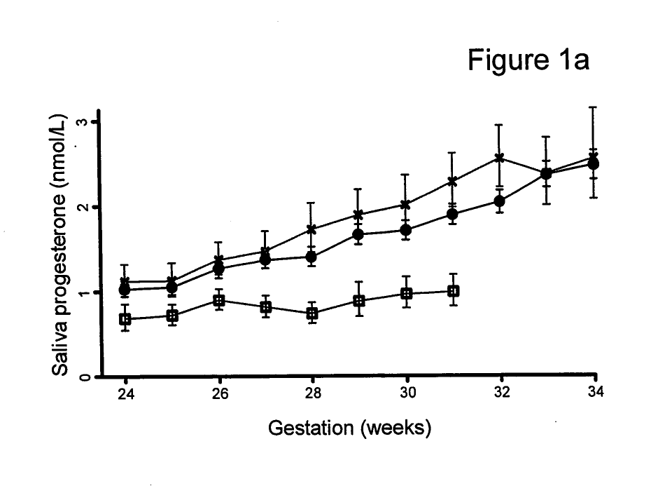

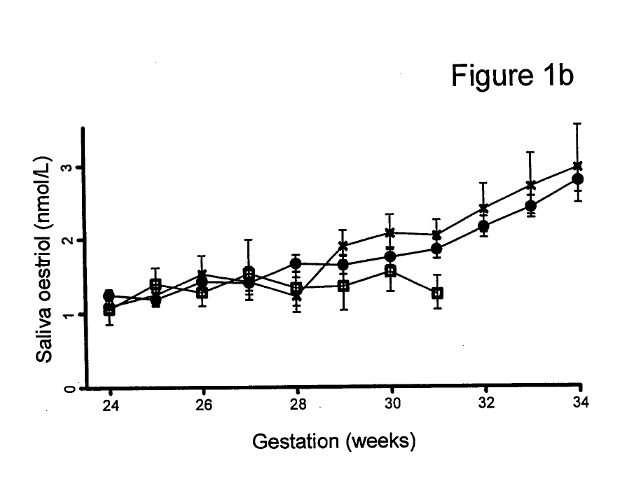

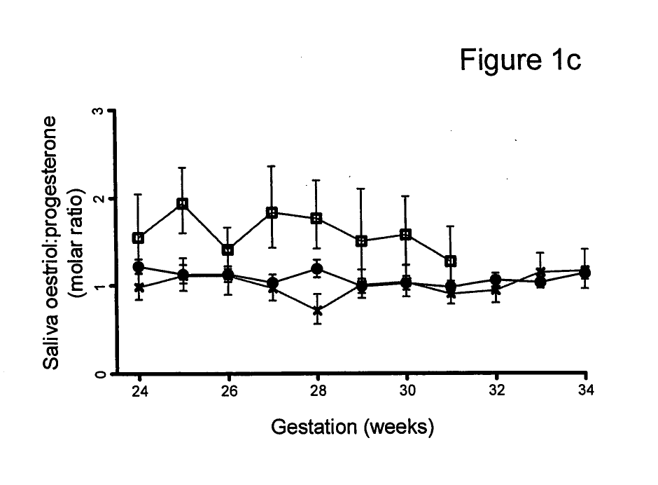

FIG. 1 shows longitudinal determination of saliva progesterone (a) and saliva oestriol (b) and the saliva oestriol:progesterone ratio (c) in women at known risk of preterm labour and delivery who delivered before 34 weeks of gestation (n=12 [symbol] ), between 34 and 37 weeks of gestation (n=16 [symbol] ) and after 37 weeks of gestation (n=64 [symbol] ). Saliva progesterone was significantly different between those who delivered before 34 weeks and those delivering between 34 and 37 weeks (p=0.007) and at term (p=0.009). There was no significant difference between groups in oestriol, but the ratio was higher in the early preterm (<34 weeks) group than those delivering between 34 and 37 weeks (p=0.041) and at term (p=0.047). Values shown are geometric means with standard error bars.

### EXAMPLES

A group of women at increased risk of preterm delivery participating in a study to assess the potential benefit of metronidazole in the prevention of preterm delivery (The PREMET study') provided saliva specimens from 24 weeks of gestation until 34 weeks or delivery in order to interrogate further the association between the steroid hormone profile and gestational age at delivery.

### Methods

In the PREMET study 892 women with singleton pregnancies, who had at least one risk factor for preterm delivery, were recruited between 23⁺⁰ and 24⁺⁶ weeks over a four and a half year period.¹ Eligibility included singleton pregnancy with a history of preterm birth or preterm prelabour rupture of the membranes before 37 completed weeks of gestation, previous late miscarriage, uterine anatomical abnormality, cervical surgery prior to the index pregnancy or current cervical cerclage. Women were screened for cervico-vaginal fetal fibronectin (fFN) at 24 and 27 weeks gestation. Those who were fFN positive (≧50 ng/mL) were randomised to receive a one week course of metronidazole or placebo.

Weekly saliva samples, provided from 24 weeks of gestation until 34 weeks or delivery, were analysed for saliva E3 and progesterone by radioimmunoassay as previously described.³ Briefly, aliquots (100 μL) of saliva were mixed with 50 μL sodium carbonate solution pH 10.5 and extracted with 10 volumes of diethyl ether. The mixture was frozen, the ether decanted and evaporated and the residue dissolved in 500 μL phosphate buffered saline (PBS); 2004 aliquots of PBS were incubated with either tritium labelled E3 (NEN) and sheep antiE3 antiserum (Bioclin Services, Cardiff UK), for E3 assays, or tritium labelled progesterone and sheep antiprogesterone antiserum, for progesterone assays. Bound and free fractions were separated using dextran-coated charcoal. Interassay coefficients of variation for two control saliva pools were 7.2 and 5.8% for E3 concentrations of 0.96 and 2.28 nmol/L and 8.1 and 6.5% for progesterone concentrations of 1.15 and 2.43 nmol/L.

### Statistical Analysis.

After consideration of distributional plots, concentrations of progesterone, E3 and the E3:progesterone ratio were log-transformed for Normality. Regression models adjusted for weekly changes with gestation were fitted in Stata (version 9.2, StataCorp, College Station, Tex.). Generalised estimating equations were used, with standard errors adjusted for heterogeneity and non-Normality using the sandwich estimator. Overall tests for differences between groups were used before comparing individual groups.

ROC (receiver operator characteristic curve) areas were estimated using standard methods based on sensitivity and specificity. Logistic regression, with the modifications described above, was used to consider the combined role of saliva progesterone and a positive fFN test as predictors of early labour. Absolute values of fFN and saliva progesterone at the same gestation were compared using Spearman's rank correlation.

### Results

One hundred and eleven women supplied saliva samples for analysis between 24 and 34 weeks of gestation. Sixty four of whom delivered at term—at or after 37 completed weeks of gestation (mean 39.7, SD 2.0); the mean number of samples provided per woman was 10.8. Twenty eight women who delivered before 37 weeks of gestation (mean 34.4, SD 2.6), following the spontaneous onset of labour, provided an average of 7.2 samples. Nineteen women were excluded from the analysis (4 withdrew consent after a positive fFN test; 5 of those delivering at term provided inadequate samples; 1 was treated with progesterone; 7 had a caesarean section before the onset of labour; 1 underwent induction of labour at 36 weeks of gestation and for 1 the outcome of pregnancy was unknown).

Nine women delivering before 37 weeks of gestation and eight delivering at or after 37 weeks of gestation were treated with metronidazole. There was no difference in the concentrations of saliva steroids between women randomised to metronidazole or placebo in either the term or preterm groups. Although metronidazole treatment was associated with a small but significant increase in preterm delivery in the PREMET study, there was no significant difference in gestational age at delivery between the 28 women randomised to treatment or placebo in the subgroup providing saliva samples for the purposes of this study (36.4 weeks in 7 women on placebo, 35.6 in 17 on metronidazole, p=0.73). Amongst this subgroup of PREMET trial participants, the predominant risk factors for entry were previous preterm delivery and previous preterm rupture of the membranes. There were no significant differences in age, ethnicity, socio-demographics or obstetric history in the women who delivered preterm (before 37 weeks) compared with those who delivered after 37 weeks of gestation.

There was no significant difference in the concentration of saliva E3 or progesterone or in the E3: progesterone ratio between women who delivered before 37 weeks of gestation or those who delivered at term. In women who delivered before 37 weeks of gestation the progesterone concentration was 89% (CI 68% to 115%, p=0.370) of the value in the women delivering at term; E3 was 99% (82% to 121%, p=0.934), and the ratio was 111% (85% to 145%, p=0.454), based on ratios of geometric means.

Delivery before 34 weeks is associated with greater morbidity than delivery between 34 and 37 weeks and has been suggested to be of differing aetiology. Women who delivered before 34 weeks of gestation, between 34 and 37 weeks of gestation and at term (>37 weeks of gestation) were therefore considered separately. Systematic comparisons of the 3 groups showed a significant difference in the saliva progesterone concentrations between the three groups (<34 weeks, 34-37 weeks and term) but not in the E3 concentration or in the E3:progesterone ratio between groups (progesterone χ²=8.26, P=0.016; E3 χ²=0.22, P=0.896; ratio x²=4.63, P=0.099).

The gestational profiles between 24 and 34 weeks for saliva progesterone, E3 and the E3:progesterone ratio are shown in FIG. 1. E3 and progesterone increased with gestation in women delivering after 37 weeks of gestation (FIG. 1a, b). The saliva progesterone concentrations in the 12 women who delivered before 34 weeks (mean 30.7 weeks, SD 2.0) following the spontaneous onset of preterm labour was significantly lower than those of the term group (63%, 95% CI 45% to 89%, n=64, p=0.09) or those delivering after the spontaneous onset of labour between 34 and 37 weeks of gestation (57%, CI 37% to 86%, n=16, p=0.007) (FIG. 1a). The E3:progesterone ratio was higher in the women who went into spontaneous labour and delivered before 34 weeks than in those delivering between 34 and 37 weeks of gestation (161%, 95% CI 102% to 206%, p=0.041) or at term (148%, 95% CI 100.4% to 217%) (FIG. 1c). In each case, results for the women delivering between 34 and 37 weeks; mean 35.8 weeks, SD 0.92) were similar to those delivering at term.

Considered alone, low saliva progesterone was a predictor of early preterm labour and delivery (before 34 weeks), compared to later delivery, with ROC areas between 0.64 and 0.69. When saliva progesterone at 24 weeks was used in conjunction with the fFN result, there was a non significant increase in the ROC area from 0.71 for fFN alone to 0.79. There was no correlation between saliva progesterone and the fFN test result (Spearman's rank correlation=−0.159 at 24 weeks, +0.093 at 27 weeks).

Maximum saliva values before 30 weeks had ROC areas 0.88 (95% Confidence Interval 0.71 to 1.00) for delivery before 30 weeks, 0.81 (0.68 to 0.93) for delivery before 34 weeks, and 0.63 (0.51 to 0.75) for delivery before 37 weeks. The sensitivity and specificity for predicting delivery before 34 weeks were 79% (49 to 95) and 53% (42 to 64) with a 10% reduction threshold, 71% (42 to 92) and 69% (58 to 78) with a 25% threshold, and 57% (29 to 82) and 84% (75 to 91) with a 50% threshold.

### Discussion

The data from this study, which has shown lower concentrations of saliva progesterone in women at known risk of preterm birth who deliver before 34 weeks of gestation, add strength to the concept that measurement of saliva steroids could play a role in early identification of a subgroup of women at risk of preterm labour.

The concentrations of progesterone and oestriol, and the gestational profile were similar in the high risk women who delivered after 37 weeks of gestation to those we have previously described in a cohort of 20 normal pregnant women providing saliva samples twice a week from 18-28 weeks of gestation and then daily until delivery.³

The lower progesterone concentration and higher E3: progesterone ratio in the women who delivered before 34 weeks supports the theory that imbalance between these hormones may be associated with preterm labour in some women. To our knowledge lower saliva concentrations of progesterone or a higher E3: progesterone ratio in women at risk of preterm labour have not been described previously. The absence of relationship with the concentration of cervico-vaginal fFN may suggest a different aetiology and the potential for identification of a subgroup of women at risk. The lower saliva progesterone concentrations and higher ratio in the early compared with the late preterm labour groups also adds strength to the suggestion that early preterm delivery may be mechanistically different from late preterm spontaneous labour and delivery. We are aware of only one study which has addressed the relationship between saliva progesterone and preterm labour. Klebanoff et al⁶ measured baseline saliva progesterone and oestriol in 386 and 413 high risk women taking part in a randomised controlled trial of 17α hydroxyprogesterone caproate which showed that the intervention had significant benefit. They found no significant association of progesterone measured at 16 to 20 weeks of gestation and outcome. Although longitudinal samples were provided by 20 women who delivered preterm and 20 who delivered at term in the placebo group, no analysis in relation to outcome was presented. As noted by these authors, the saliva progesterone concentrations measured between 16 and 20 weeks of gestation may not reflect changes occurring later in pregnancy.

Progesterone is essential for the maintenance of pregnancy and promotes uterine relaxation by suppressing the formation of gap junctions and reducing prostaglandin synthesis and the concentration of oxytocin receptors in the myometrium. Progesterone also increases cellular calcium binding and may lower myometrial intracellular calcium.³ Progesterone also has anti-inflammatory properties², and lower than normal progesterone status, as identified in the early preterm group in the present study, could contribute to an exaggerated response to bacterial invasion, widely recognised as a cause of early preterm labour. Because of promising data from clinical trials, progestogen supplementation is now being widely investigated as a preventative intervention in women at risk of preterm labour and it is suggested that anti-inflammatory properties of progesterone may also contribute to the potential benefit.

On the basis of the data presented in this study, further prospective studies with larger numbers of women are now indicated to assess whether measurement of saliva oestriol and progesterone in women at risk for preterm labour could help to select those most likely to respond to progesterone treatment.

### CONCLUSIONS

Saliva progesterone concentrations between 24 and 34 weeks of gestation were significantly lower in women who delivered spontaneously before 34 weeks of gestation than in those delivering after 37 weeks. Given the likely heterogeneous origins of preterm delivery, it is unlikely that a single test will serve to identify all women at risk, and that an algorithm which combines clinical risk factors and biomarkers is likely to provide better accuracy. We suggest that future investigations should address the potential value of a combination of tests, including measurement of fFN and oestriol and progesterone concentrations, for the prediction of preterm labour and to determine whether saliva progesterone measurement can be used as an aid to identification of women who would benefit from progesterone treatment.

### REFERENCES

1. Shennan A, Crawshaw S, Briley A, Hawken J, Seed P, Jones G et al. A randomised controlled trial of metronidazole for the prevention of preterm birth in women positive for cervicovaginal fetal fibronectin: the PREMET study. BJOG 2006; 113:65-74.
2. Zakar T, Hertelendy F. Progesterone withdrawal: key to parturition. Am J Obstet Gynecol 2007; 289-296.
3. Dame J, McGarrigle H H G, Lachelin G C L. Saliva oestriol, oestradiol, oestrone and progesterone levels in pregnancy: spontaneous labour at term is preceded by a rise in the saliva oestriol:progesterone ratio. BJOG 1987; 94:227-235.
4. Dame J, McGarrigle H H G, Lachelin G C L. Increased saliva oestriol to progesterone ratio before idiopathic preterm delivery: a possible predictor for preterm labour? BMJ 1987; 294:270-273.
5. Mc Gregor J A, Jackson G M, Lachelin G C L, Goodwin T M, Artal R, Hastings C et al. Saliva estriol as risk assessment for pretem labor. Am J Obstet Gynecol 1995; 173:1337-1342.
6. Klebanoff M A, Meis P J, Dombrowski M P, Zhao Y, Moawad A H, Northern A et al. Saliva progesterone and estriol among pregnant women treated with 17-α-hydroxyprogesterone caproate or placebo Am J Obstet Gynecol 2008; 199: 506 el-7.

1. A method for identifying whether a pregnant subject has an increased risk of preterm labour comprising:
measuring the progesterone concentration in a sample obtained from the pregnant subject, wherein a reduced progesterone concentration is indicative of an increased risk of preterm labour and delivery.

2. A method according to claim 1, wherein the pregnant subject is a mammal.

3. A method according to claim 2, wherein the mammal is a human.

4. A method according to claim 3, wherein the reduced progesterone concentration indicates that the human is at risk of labour before 37 weeks' completed gestation.

5. A method according to claim 3, wherein the reduced progesterone concentration indicates that the human is at risk of labour before 34 weeks' completed gestation.

6. A method according to any of claims 1-5, wherein the sample is a saliva sample.

7. A method according to claim 1, further comprising comparing the sample with a comparable sample obtained from the pregnant subject at an earlier date.

8. A method according to claim 1, further comprising comparing the sample with one or more samples obtained from other pregnant subjects at the same stage of gestation.

9. A method according to claim 1, further comprising testing the sample, or other samples obtained from the pregnant subject for other indicators of a risk of preterm labour.

10. A method of treating a subject at risk of preterm labour comprising:
identifying the subject as being at risk of preterm labour using the method of claim 1; and
administering progesterone to the subject in a therapeutically effective amount.

11. A method to prevent or delay preterm labour, comprising:
administering progesterone to a subject at risk of preterm labour, the subject having been tested and identified as being at risk of preterm labour by virtue of a reduced progesterone concentration in a sample obtained from the subject relative to a comparable sample obtained from the subject at an earlier date or relative to a comparable sample obtained from other subjects at the same stage of gestation.

12. The method of claim 10 wherein the sample is a saliva sample.

13. The method of claim 11 wherein the samples are saliva samples.
